(19) United States
(12) Patent Application Publication
(10) Pub. No.: US 2008/0138388 A1
(43) Pub. Date: Jun. 12, 2008

(54) Transdermal Absorption Patch

(75) Inventors: Kazunosuke Aida, Ibaraki (JP); Yasunari Michinaka, Ibaraki (JP); Takaaki Terahara, Ibaraki (JP)

Correspondence Address: EDWARDS ANGELL PALMER & DODGE LLP, P.O. BOX 55874, BOSTON, MA, 02205, US

(73) Assignee: Hisamitsu Pharmaceutical Co., Inc., Saga (JP)

(21) Appl. No.: 11/883,672

(22) PCT Filed: Jan. 24, 2006
(86) PCT No.: PCT/JP2006/300994

§ 371 Date: Aug. 2, 2007

(30) Foreign Application Priority Data
Feb. 4, 2005 (JP) 2005-028550

(51) Int. Cl. A61K 9/70; A61K 31/445; A61P 43/00

(52) U.S. Cl. 424/448; 514/319

(57) ABSTRACT

It is intended to provide a transdermal absorption patch which is highly excellent in transdermal absorption properties and long-lasting drug effect even in the case where a drug-effect component contained in the transdermal absorption patch is a basic drug hardly soluble in a pressure-sensitive adhesive base, has a high stability of the drug contained therein with the passage of time and can achieve improvement in the compliance and simplification of the administration method. These problems can be solved by providing a transdermal absorption patch which contains a basic drug having an octanol/water partition coefficient (logarithm) in the free state of 3 or above, a pressure-sensitive adhesive base and a (meth)acrylic copolymer having carboxyl group.

### TECHNICAL FIELD

The present invention relates to a transdermal absorption patch containing a (meth)acrylate copolymer with excellent transdermal absorption properties and sustainability of drug efficacy.

### BACKGROUND ART

As an administration method of drugs, conventionally oral administration methods which use tablets, capsules and syrups are known. However, oral administration has several disadvantages including the first pass effect at the liver after the drug was absorbed, and that a higher-than-required blood concentration of the drug is temporarily observed after administration. In addition, in the oral administration, a number of side effects such as gastrointestinal disorders, nauseousness, and loss of appetite have been reported. Accordingly, with the aim of solving such problems in the oral administration and of development of preparations which can be taken by patients easily, safely and continually, the development of transdermal absorption patches has been actively promoted in recent years. Administration methods using patches can solve the above-mentioned various problems in the oral administration methods, and have advantages such as elongation of the duration of drug action, a decrease in the number of times of administration, improvement of compliance, and easiness of administration and easiness of discontinuation; moreover, they are also effective in elderly patients and infant patients; thus, the use of patches is expected as an remarkably useful administration method.

However, because the stratum corneum layer has a very high fat solubility, skin permeability of drugs is generally low, and the stratum corneum layer of the normal skin has a barrier function to prevent invasion of exogenous materials; therefore, when a conventional adhesive patch is used, in many cases a drug blended therein is not sufficiently absorbed transdermally.

Therefore, to increase transdermal absorption properties of a drug in the transdermal administration using adhesive patches, various studies on composition and others of adhesive patches have been carried out. Transdermal absorption patches generally consist of an adhesive matrix (adhesive layer), a backing, and a release liner, wherein the adhesive matrix contains a drug, an adhesive base and other additives. Accordingly, development of the composition of adhesive bases to enhance transdermal absorption of drugs have been promoted, and adhesive patches wherein a polymer material is used as the adhesive base have been proposed; the polymer material includes acrylic polymers such as an acrylic ester copolymer, rubber polymers such as a styrene-isoprene-styrene block copolymer, polyisobutylene, a natural rubber, and silicone polymers such as polydimethylsiloxane (refer to Patent documents 1-3). Since these adhesive bases were lipophilic, it became possible to dissolve and blend a relatively large amount of a drug in the adhesive base by means of further addition of a solubilizing agent (Patent documents 4-7), so that fairly good transdermal absorption properties could be realized in certain drugs.

However, when a basic drug is used, the transdermal absorption was not sufficient even with the above attempts. Namely, in order to effectively exert the drug efficacy, it is necessary to increase the transdermal absorption rate of a drug by increasing the drug concentration in the adhesive base; however, some basic drugs have poor solubility in adhesive bases that are generally lipophilic, so that sufficient transdermal absorption properties cannot be obtained with such drugs. Moreover, with the adhesive patches using conventional adhesive bases, physical properties such as adhesiveness as well as time course stability of drugs blended in the adhesive patches may have a problem. Thus, further improvement of the preparations has been desired.

Meanwhile, in recent years, with an increase in the number of elderly people, an increase in the number of patients with cognitive deficiencies such as Alzheimer's disease has been noted as an important issue in the field of medical care. In this regard, development of anti-Alzheimer's disease drugs has been promoted, and therapeutic application of various drugs including donepezil hydrochloride, memantine, galantamine and rivastigmine has been attempted domestically as well as internationally. In particular, donepezil hydrochloride has already been placed on the market as tablets and fine granules, exhibiting useful therapeutic effects. However, for patients having symptoms in a progressed stage, it is sometimes difficult to take oral agents such as tablets and fine granules. In addition, considering that the number of elderly patients with deteriorated swallowing function is increasing, and that there are reports of side effects in the digestive organs such as peptic ulcer, duodenal ulcer and bleeding in the digestive tract, a preparation for transdermal administration of anti-Alzheimer's disease drugs including donepezil hydrochloride has been proposed to remedy such situations (Patent documents 8 and 9).

Actually, however, in order to effectively and transdermally administer a drug such as donepezil hydrochloride as a preparation having sufficient transdermal absorption properties and sustainability of drug efficacy, it is necessary to contain the drug in an adhesive layer at a high concentration in a stable manner; however, such a drug has a low solubility in the adhesive bases as mentioned above, and even when the drug dissolves transiently, it cannot stay in the adhesive layer stably due to its crystallization and other reasons after production of preparations Thus, to produce preparations containing such a drug at a high concentration in a stable manner in an adhesive layer was extremely difficult. Accordingly, to date there is no transdermal preparation of anti-Alzheimer's disease drug placed on the market, having sufficient transdermal absorption properties, drug-efficacy sustainability and time course stability which can be used for actual therapy of patients.

### [Patent document 1] WO02/038139

### [Patent document 2] JP A No. 5-39222

### [Patent document 3] JP A No. 5-194201

### [Patent document 4] JP No. 3496169

### [Patent document 5] JP No. 3021661

### [Patent document 6] JP A No. 2004-528359

### [Patent document 7] JP A No. 2004-529891

### [Patent document 8] JP A No. 11-315016

### [Patent document 9] WO03/032960

### DISCLOSURE OF THE INVENTION

### Problem to be Solved by the Invention

Therefore, a problem to be solved by the present invention is to provide a transdermal absorption patch having excellent transdermal absorption properties and sustainability of drug efficacy even when a medicinal ingredient contained in the transdermal absorption patch is a basic drug hardly soluble in an adhesive base, which also shows a time-course stability of the drug in the preparation, and is able to improve compliance and to simplify the administration method. Moreover, another problem to be solved by the invention is to provide a donepezil-containing adhesive patch having sufficient transdermal absorption properties, drug-efficacy sustainability and time course stability, which enables actual therapy of patients.

### Means of Solving the Problem

During extensive research to solve the above problems, the inventors found that, in an adhesive base, by containing a basic drug having the logarithm of octanol-water partition coefficient (logP) in the free state of 3 or more which shows low solubility in the adhesive base, and by further containing a (meth)acrylate copolymer having a carboxyl group, said drug can be stably dissolved in the adhesive base, thereby making a transdermal absorption patch having remarkably excellent transdermal absorption properties and sustainability of drug efficacy as well as superior time course stability and superior physical properties of the preparation; and the inventors completed the invention. The inventors further advanced the research, and found for the first time that by blending donepezil in an adhesive patch of the invention as said basic drug, the adhesive patch containing the high-concentration of donepezil and having superior time course stability and physical properties can be realized, and that this adhesive patch can exert very good transdermal absorption of the donepezil, thus completing the invention.

Namely, the present invention relates to a transdermal absorption patch containing a basic drug having a logarithm of octanol-water partition coefficient in the free state of 3 or more, an adhesive base, and a (meth)acrylate copolymer having a carboxyl group.

The invention also relates to said transdermal absorption patch, wherein the adhesive base is an acrylic adhesive.

The invention furthermore relates to said transdermal absorption patch, wherein the acrylic adhesive is a polymer having a carboxyl group or a hydroxyl group.

Furthermore, the invention relates to said transdermal absorption patch, wherein the (meth)acrylate copolymer having a carboxyl group is a copolymer consisting of methacrylic acid and methyl methacrylate.

The invention also relates to said transdermal absorption patch, further containing a fatty acid and/or an aliphatic alcohol having a carbon number of 8 or more.

The invention furthermore relates to said transdermal absorption patch, wherein the basic drug has a low lipophilicity. The invention also relates to said transdermal absorption patch, wherein the basic drug has a low water solubility.

The invention also relates to said transdermal absorption patch, wherein the basic drug is donepezil.

Moreover, the invention relates to said transdermal absorption patch, wherein an amount of the basic drug is 5 wt % or more relative to a total weight of the adhesive layer.

### EFFECTS OF THE INVENTION

With the adhesive patch of the invention, by containing an adhesive base and a (meth)acrylate copolymer having a carboxyl group in an adhesive layer of the adhesive patch, a basic drug having the logarithm of octanol-water partition coefficient (logP) in the free state of 3 or more can be completely dissolved in the adhesive base and said basic drug can be stably contained without occurrence of crystallization after production of preparations. In addition, for basic drugs having a logP value of 3 or more, even when their solubility in adhesive bases is low, the basic drugs can be blended in a stable dissolution state into adhesive bases at high concentrations. Accordingly, good skin permeability and effect-sustainability of the drug can be achieved simultaneously, so that preparations having remarkably superior transdermal absorption properties, which sufficiently exert efficacy of the drug contained in the patches, can be realized. Moreover, this skin permeability and effect-sustainability can be further improved by further addition of a fatty acid and/or an aliphatic alcohol having a carbon number of 8 or more.

Furthermore, the adhesive patch of the invention has superior physical properties such as adhesiveness, and it can exist in an remarkably stable manner over time without deposition due to crystallization of the drug in the preparations; thus the invention can provide a drug-containing adhesive patch with a high level of safety and reduced skin irritation. In addition, when a drug which has been conventionally administered as an oral preparation is administered by the adhesive patch of the invention, the drug can be administered as a preparation with significantly superior sustainability of the drug efficacy, without being subjected to decomposition in the digestive organs and metabolism in the liver and others; thus, the adhesive patch of the invention can simplify conventional dosing methods and improve compliance.

Moreover, by blending donepezil as a drug in the adhesive patch of the invention, the donepezil-containing adhesive patch having the above-mentioned effects can be provided. Namely, the donepezil-containing anti-Alzheimer's disease preparation of the invention has superior skin permeability, so that, according to the invention, donepezil-containing transdermal absorption preparations with remarkably superior transdermal absorption properties that may improve conventional dosing methods and compliance can be provided.

As such, the present inventive patch enables, even when a basic drug having a low solubility in adhesive bases and the logarithm of octanol-water partition coefficient (logP) in the free state of 3 or more is used in the adhesive patch, the drug blended in the adhesive patch can be sufficiently and transdermally absorbed and the drug efficacy can be sufficiently exerted, the present inventive patch also having superior physical properties and superior time course stability of the drug blended in the preparation; furthermore, a donepezil-containing adhesive patch having the above-mentioned effects can be realized by blending donepezil. The adhesive patch having such effects has been realized for the first time by the invention.

### BEST EMBODIMENT FOR CARRYING OUT THE INVENTION

Hereinafter, embodiments of the invention are illustrated in detail.

The adhesive patch of the invention typically consists of an adhesive matrix (adhesive layer), a backing, and a release liner, wherein the adhesive layer contains a basic drug having the logarithm of octanol-water partition coefficient (logP) in the free state of 3 or more, an adhesive base and a (meth)acrylate copolymer having a carboxyl group; other additives such as an adsorption enhancer may be added to the adhesive layer.

The adhesive patch of the invention is effective for basic drugs having the logarithm of octanol-water partition coefficient (logP) in the free state of 3 or more; among such basic drugs, it is particularly effective for those having a low oil solubility and low water solubility. The oil solubility and water solubility of drugs can be quantitatively expressed using “logarithm of octanol-water partition coefficient (logP)” as an index. The “logarithm of octanol-water partition coefficient (logP)” of the drug (logPow) is obtained in a dilute solution of the drug as the logarithm of the ratio (Pow) of the solubility in n-octanol to the solubility in water.

The value of logP depends on both the solubility in n-octanol and solubility in water of the drug; in other words, it depends on both the oil solubility and water solubility. Accordingly, drugs with large logP values include, conceptually, “drugs having high oil solubility,” “drugs having oil solubility and poor water solubility,” “drugs having low oil solubility and low water solubility,” and “drugs having remarkably high oil solubility and slight water solubility;” and the transdermal absorption patch of the invention is effective with any of these basic drugs having such characteristics. Among the basic drugs having high logP values, when those having a logP value in the free state of 3 or more with low oil solubility and low water solubility are used, the present invention can exhibit particularly excellent effects in improving the solubility in lipophilic adhesive bases and improving the skin permeability.

In the invention, “low oil solubility” means that the oil solubility of a drug is low; the oil solubility of a drug can be determined by, for example, an amount of octanol required to dissolve 1 g of the drug as an index. Drugs with low oil solubility of the invention include, although not specifically limited, preferably those requiring 30 ml or more of octanol for complete dissolution of 1 g of said drug in the free state, and more preferably those requiring 10 ml or more of octanol.

Furthermore, in the invention, “low water solubility” means that the drug is hardly soluble in water; the water solubility of a drug can be determined by, for example, an amount of water required to dissolve 1 g of the drug as an index. Drugs with low water solubility of the invention include, preferably those requiring 100 ml or more of water for complete dissolution of 1 g of said drug in the free state, and more preferably those requiring 1000 ml or more of water, and furthermore preferably those requiring 10000 ml or more of water.

Thus, the basic drug used in the present invention is not particularly limited so long as it is the one having the above logP value in the free state of 3 or more. The logP value of preferred drugs used in the inventive patch is preferably 3 or more, more preferably between 3 and 5, and most preferably between 4 and 5. Specific examples of preferred drugs having such logP values include pergolide (logP=4.07), oxybutynin (logP=4.28), pridinol (logP=3.79), tamsulosin (logP=3.22), ambroxol (logP=3.00), trandopril (logP=4.00), and donepezil (logP=4.71); among them, donepezil is particularly preferred. Only one kind of such drugs may be used, or a combination of two or more kinds may be used.

Meanwhile, in the invention, whereas the logP value in the free state of each drug is used as a standard, pharmaceutically acceptable acid-addition salts and base-addition salts of the basic drug having a logP value in the free state of 3 or more may also be used; accordingly, the drug can be present in a state of acid-addition salt or base-addition salt in the adhesive layer, and it can also be present in the free state. Pharmaceutically acceptable salts are not particularly limited, which may be inorganic or organic salts. Specific suitable examples of the above preferred basic drug include pergolide mesylate, oxybutynin hydrochloride, pridinol mesylate, ambroxol hydrochloride, tamsulosin hydrochloride, and donepezil hydrochloride, etc.

In the adhesive patch of the invention, the basic drug having a logP value in the free state of 3 or more is blended at an amount of preferably 5-40 wt %, more preferably 10-40 wt %, and particularly preferably 20-40 wt % relative to the weight of the entire composition of the adhesive layer. This is because when the amount is less than 5 wt %, the resulting patch tends to lack transdermal absorption properties so that sufficient drug efficacy cannot be achieved; whereas when the amount exceeds 40 wt %, then the resulting patch tends to have insufficient adhesiveness as an adhesive patch.

The adhesive base used in the transdermal absorption patch of the invention is not particularly limited, so long as it can be used as a base of the adhesive layer, which includes, for example, hydrophobic (lipophilic) polymers such as acrylic adhesives, styrene-isoprene-styrene block copolymers, isoprene rubber, polyisobutylene, styrene-butadiene-styrene block copolymers, styrene-butadiene rubber, natural rubber, polydimethylsiloxane. Amongst all, an acrylic adhesive is especially preferred.

The acrylic adhesive used in the inventive adhesive patch is preferably a polymer consisting mainly of acrylic esters, wherein, as the acrylic esters, one or more (meth)acrylic C₂₋₁₈ alkyl esters are used as major components, and wherein said polymer is obtained by copolymerization of said acrylic esters and optional one or more copolymerizable monomers (for example, 2-ethylhexyl acrylate, vinyl pyrrolidone, vinyl acetate, methoxyethyl acrylate, hydroxyethyl acrylate and acrylic acid, etc.). (Meth)acrylic C₂₋₁₈ alkyl esters herein refer to esters obtained from primary to tertiary alcohols having a carbon number in the alkyl group of 2-18, preferably 4-12, and an acrylic acid or methacrylic acid.

As the acrylic adhesive, those having a carboxyl group or hydroxyl group are preferred. This is because by the use of an adhesive having a carboxyl group or hydroxyl group, solubility of a basic drug in the adhesive can be improved, so that transdermal absorption properties can be improved.

Specific examples of the acrylic adhesive used in the inventive adhesive patch include Duro-Tak87-2516, Duro-Tak87-4098, Duro-Tak87-2194 (acrylic acid/vinyl acetate copolymer (National Starch & Chemical Company)), and TSR (2-ethylhexyl acrylate-vinyl pyrrolidone copolymer (Sekisui Chemical Co., Ltd.)). These adhesive bases may be used alone or in a combination of two or more thereof. The amount of blending of the adhesive base is, considering the formation of the adhesive layer and the skin permeability of active components, preferably 10-80 wt %, more preferably 20-75 wt %, and particularly preferably 40-65 wt % relative to the weight of the entire composition of the adhesive layer.

The (meth)acrylate copolymer having a carboxyl group used in the inventive adhesive patch is not particularly limited so long as it is a copolymer of an acrylic acid or methacrylic acid and a methacrylate alkyl ester or acrylate alkyl ester (such as methyl ester, ethyl ester, propyl ester and butyl ester). All of these copolymers are non-adhesive solid having a carboxyl group but compatible to adhesive bases and are present in a dissolved state in the adhesive patches. By blending such (meth)acrylate copolymer having a carboxyl group into the adhesive base, even a basic drug having a logP value of 3 or more and a low solubility in the adhesive base can be dissolved into the adhesive base at a high concentration, and transdermal absorption properties can simultaneously be significantly improved. In addition, blending the (meth)acrylate copolymer having a carboxyl group can further improve skin permeability by the addition of absorption enhancers such as fatty acids and aliphatic alcohols. Moreover, when the (meth)acrylate copolymer having a carboxyl group is used, the resulting preparation is not plasticized even when an absorption enhancer such as a fatty acid or aliphatic alcohol which tends to plasticize the adhesive layer is blended, and good adhesiveness can be obtained. Furthermore, by blending the (meth)acrylate copolymer having a carboxyl group, the basic drug blended into the preparation can stably exist in the adhesive layer without crystals being separated for a long period after production of the preparation. Since these effects cannot be obtained when a (meth)acrylate copolymer without a carboxyl group is used, it is considered that the significant improvement of transdermal absorption properties, improvement of dissolution, and prevention of precipitation of the basic drug, as well as prevention of plasticization of the adhesive layer are obtained due to the action of the carboxyl group which is contained in the (meth)acrylate copolymer-blended.

Among (meth)acrylate copolymers having a carboxyl group, particularly preferred copolymers used in the inventive adhesive patch are metacrylate copolymers consisting of methacrylic acid and methyl methacrylate. Preferred specific examples of such metacrylate copolymers include Eudragit L (major component=methacrylic acid 38-52%, copolymerization component=methyl methacrylate), Eudragit S (major component=methacrylic acid 25-34.5%, copolymerization component=methyl methacrylate) and others.

In addition, only one kind of such (meth)acrylate copolymers may be used, or a combination of two or more kinds may be used. In the adhesive patch of the invention, the (meth)acrylate copolymer is blended at an amount of 1 wt % or more, preferably 1-20 wt %, and more preferably 5-10 wt % relative to the weight of the entire composition of the adhesive layer. This is because when the amount is less than 1 wt %, the solubility of the drug tends to be insufficient, and when the amount exceeds 20 wt %, the adhesiveness as an adhesive patch tends to decrease.

In the adhesive patch of the invention, in addition to the above essential components (an basic drug having the logarithm of octanol-water partition coefficient in the free state of 3 or more, an adhesive base, and a (meth)acrylate copolymer having a carboxyl group), an absorption enhancer may be blended to increase the transdermal absorption properties of the medicinal ingredients. Examples of the absorption enhancer used in the invention include fatty acids having a carbon number of 6-20, aliphatic alcohols, fatty acid esters or ethers or amides, aromatic organic acids, aromatic alcohols, aromatic organic acid esters or ethers (those heretofore described may be either saturated or unsaturated, and either cyclic, straight chain or branched), furthermore, lactic acid esters, acetic acid esters, monoterpene compounds, sesquiterpene compounds, Azone, Azone derivatives, glycerin fatty acid esters, sorbitan fatty acid esters, polyethylene glycol fatty acid esters, polyoxyethylene alkyl ethers, polyoxyethylene hardened castor oil (HCO), sucrose fatty acid esters and the like. Among them, fatty acids having a carbon number of 8 or more (such as caprylic acid, capric acid, myristic acid, palmitic acid, oleic acid, stearic acid, etc.), and aliphatic alcohols (such as oleyl alcohol, isostearyl alcohol, lauryl alcohol, octyl alcohol, decyl alcohol, myristyl alcohol, cetyl alcohol, stearyl alcohol, etc.) are particularly preferred, because they significantly improve the transdermal absorption properties of the inventive adhesive patch when they are blended in the adhesive layer composition of the invention together with a (meth)acrylate copolymer having a carboxyl group.

These absorption enhancers may be used alone or in a combination of two or more thereof. The amount of blending of an absorption enhancer is, taking into consideration the sufficient permeability of active components into the skin and skin irritation as adhesive patches, preferably 1-10 wt %, more preferably 2-8 wt %, and especially preferably 3-6 wt % relative to the weight of the entire composition of the adhesive layer.

In the adhesive layer of the inventive adhesive patch, when the adhesive force is insufficient, a tackifier resin may further be blended. Tackifier resins which can be used include rosin derivatives (such as rosin, glycerin esters of rosin, hydrogenated rosin, glycerin esters of hydrogenated rosin, pentaerythritol esters of rosin), saturated alicyclic hydrocarbon resins (such as ARKON P-100, Arakawa Chemical Industries, Ltd.), aliphatic hydrocarbon resins (such as Quintone B170, Zeon Corporation), terpene resins (such as Clearon P-125, Yasuhara Chemical), maleic acid resins and the like. Amongst all, particularly preferred tackifier resins are glycerin esters of hydrogenated rosin, saturated alicyclic hydrocarbon resins, aliphatic hydrocarbon resins, and terpene resins.

These tackifier resins may be used alone or in a combination of two or more thereof. The amount of blending of a tackifier resin is, taking into consideration the sufficient adhesive force and skin irritation upon peeling off as adhesive patches, preferably 20-60 wt %, more preferably 30-60 wt %, and especially preferably 40-60 wt % relative to the weight of the entire composition of the adhesive layer.

Furthermore, a plasticizer may be blended in the adhesive patch of the invention. Plasticizers which may be used include paraffin oil, squalane, squalene, vegetable oils (such as olive oil, camellia oil, castor oil, tall oil, arachis oil), dibasic acid esters (such as dibutyl phthalate, dioctyl phthalate, etc.), liquid rubbers (such as polybutene, liquid isoprene rubber), diethylene glycol, polyethylene glycol, glycol salicylate, propylene glycol, dipropylene glycol, crotamiton and others. Among them, liquid paraffin, liquid polybutene, glycol salicylate, and crotamiton are particularly preferred.

These plasticizers may be used alone or in a combination of two or more thereof. The amount of blending of such plasticizer is, taking into consideration the sufficient skin permeability of active components and sufficient maintenance of cohesion force as adhesive patches, preferably 5-30 wt %, more preferably 10-30 wt %, and especially preferably 10-20 wt % relative to the weight of the entire composition of the adhesive layer.

Furthermore, if necessary, other agents such as antioxidants, fillers, cross-linking agents, preservatives and ultraviolet absorbers may be blended in the adhesive patch of the invention. As antioxidants, tocopherol and its ester derivatives, ascorbic acid, ascorbic acid-stearic acid ester, nordihydroguaretic acid, dibutyl hydroxy toluene (BHT), butyl hydroxy anisole and the like are preferred. As fillers, calcium carbonate, magnesium carbonate, silicates (such as aluminum silicate, magnesium silicate, etc.), silicic acid, barium sulfate, calcium sulfate, calcium zincate, zinc oxide, titanium oxide and the like are preferred. As cross-linking agents, amino compounds, phenol compounds, epoxy compounds, isocyanate compounds, organic peroxides, metal alcoholates, metal chelates and the like are preferred. As preservatives, ethyl p-hydroxybenzoate, propyl p-hydroxybenzoate, butyl p-hydroxybenzoate and the like are preferred. As ultraviolet absorbers, p-aminobenzoic acid derivatives, anthranilic acid derivatives, salicylic acid derivatives, coumarin derivatives, amino-acid compounds, imidazoline derivatives, pyrimidine derivatives, dioxane derivatives and the like are preferred.

Such an antioxidant, filler, cross-linking agent, preservative and ultraviolet absorber may be blended at an amount of preferably 10 wt % or less, more preferably 5 wt % or less, and especially preferably 2 wt % or less relative to the entire composition of the adhesive layer of the adhesive patch.

Furthermore, in the adhesive patch of the invention, basic alkali metal hydroxides or basic alkali earth metal hydroxides such as sodium hydroxide, potassium hydroxide, calcium hydroxide, and magnesium oxide may be used as a pH adjuster. In addition, when an alkali metal hydroxide or alkali earth metal hydroxide such as sodium hydroxide and calcium hydroxide is added as a pH adjuster, the molar ratio of this substance to the basic drug is preferably 1:1-1:0.5.

The adhesive patch of the invention may be prepared by any well-known method; for example, it can be obtained in such a manner that the above essential components (a basic drug having the logarithm of octanol-water partition coefficient in the free state of 3 or more, an adhesive base, and a (meth)acrylate copolymer having a carboxyl group) are dissolved in a solvent such as toluene, hexane and ethyl acetate, and the mixture is extended on a release liner or a backing and the solvent is removed by drying, then the resultant is attached to the backing or the release liner.

The backing of the inventive adhesive patch is not particularly limited so long as it is appropriate for supporting the adhesive layer; a stretch or nonstretch material may be used. For example, fabric and non-woven fabric including polyesters such as polyethylene terephthalate, polyurethane, polyvinyl acetate, polyvinylidene chloride and polyethylene, aluminum foil and others, or composite materials thereof may be used.

Furthermore, as the release liner of the inventive adhesive patch, films such as polyesters (e.g. polyethylene terephthalate), polyvinyl chloride and polyvinylidene chloride, a laminated film of high-quality paper with polyolefin and the like may be used. In such a release liner, to facilitate the operation of releasing the release liner from the adhesive side, a fluorine treatment or silicone treatment is preferably applied on the contact side of the release liner to the adhesive layer.

### EXAMPLES

In the following, the invention is explained in more detail using examples. The invention, however, is not limited to these examples, and the sequence of blending of each component is not particularly limited. Furthermore, various modifications are possible without departing from the technical idea of the invention. Here, in Table 1 below, “%” means “wt %” unless otherwise specified.

### Fabrication of Transdermal Absorption Patches (Examples 1-6 and Comparative Examples 1-10)

All of the preparations were prepared in accordance with the formulations listed in Table 1 below on the following procedures.

### Example 1

### 1) Donepezil hydrochloride is measured and introduced into a vial container.

### 2) Methanol of equal weight to the donepezil hydrochloride is added to the container.

### 3) While stirring at a low speed (200 rpm, magnetic stirrer), a 10 N-8 N sodium hydroxide (NaOH) solution is added and stirred overnight, the amount of NaOH being equimolar to donepezil hydrochloride.

### 4) An adhesive Duro-Tak87-4098 and Eudragit L100 are measured and introduced into another vial container.

### 5) Ethyl acetate of equal amount to donepezil hydrochloride is added to 4), and the mixture is stirred for overnight (700 rpm).

6) 5) is added to 3), and the mixture is stirred for 3 h (500 rpm).
7) 75 μm of a silicone-treated polyethylene terephthalate (PET) film is coated with the composition obtained in 6) (coating is applied such that the adhesive matrix after drying becomes 100 g/m²), and dried at 100° C. for 10 min. Then, a polyethylene terephthalate film (sand-mat treated, 25 μm) as a backing is laminated on said film to obtain the adhesive patch of the invention (Example 1).

### Example 2

The adhesive patch of the invention (Example 2) is obtained similarly to Example 1 except Duro-Tak87-4098 and Eudragit L100 in Example 1 being replaced with Duro-Tak87-2516 and Eudragit S100, respectively.

### Example 3

The adhesive patch of the invention (Example 3) is obtained similarly to Example 1 except Duro-Tak87-4098 and Eudragit L100 in Example 1 being replaced with TSR and Eudragit S100, respectively, and the amount of ethyl acetate added in 5) above being 1.5 fold of donepezil hydrochloride.

### Examples 4-6

The adhesive patches of the invention (Example 4-6) are obtained similarly to Example 3 except Oleyl alcohol (Example 4), isostearyl alcohol (Example 5) or lauryl alcohol (Example 6) being measured and added together with the donepezil hydrochloride in 1) above.

### Comparative Example 1

The adhesive patch (Comparative example 1) is obtained similarly to Example 1 except Eudragit L100 of Example 1 being not added.

### Comparative Example 2

The adhesive patch (Comparative example 2) is obtained similarly to Example 1 except Eudragit L100 in Example 1 being replaced with Eudragit EPO ((meth)acrylate copolymer without a carboxyl group, methyl methacrylate/butyl methacrylate/dimethylaminoethyl methacrylate copolymer).

### Comparative Examples 3 and 4

The adhesive patches (Comparative examples 3 and 4) are obtained similarly to Example 2 except Eudragit S100 of Example 1 being not added.

### Comparative Example 5

The adhesive patch (Comparative example 5) is obtained similarly to Example 2 except Eudragit S100 in Example 2 being replaced with Eudragit EPO ((meth)acrylate copolymer without a carboxyl group, methyl methacrylate/butyl methacrylate/dimethylaminoethyl methacrylate copolymer).

### Comparative Examples 6-9

The adhesive patches (Comparative example 6-9) are obtained similarly to Comparative example 4 except Pyrothiodecane (Comparative example 6), glyceryl monolaurate (GML) (Comparative example 7), propylene glyceryl monolaurate (PGML) (Comparative example 8) or isostearyl alcohol (Comparative example 9) being measured and added together with the donepezil hydrochloride in 1) above.

### Comparative Example 10

The adhesive patch (Comparative example 10) is obtained similarly to Example 3 except Eudragit S100 of Example 3 being not added.

TABLE 1

| | | | | | | | | | Jmax | Tack | |
|---|---|---|---|---|---|---|---|---|---|---|---|
| | | | Donepezil | | | Absorption | | | (μg/ | value | |
| Example No. | Adhesive base | | hydrochloride | Eudragit | | enhancer | | NaOH | cm²/hr) | (gF) | Crystallization |
| Example 1 | Duro-Tak87- | 56.6% | 35.0% | Eudragit | 5.0% | — | | 3.4% | 15.43 | 677 | ◯ |
| | 4098 | | | L100 ⋆ | | | | | | | |
| Example 2 | Duro-Tak87- | 62.1% | 30.0% | Eudragit | 5.0% | — | | 2.9% | 13.66 | 779 | ◯ |
| | 2516 # | | | S100 ⋆ | | | | | | | |
| Example 3 | TSR | 56.6% | 35.0% | Eudragit | 5.0% | — | | 3.4% | 17.57 | 830 | ◯ |
| | | | | S100 ⋆ | | | | | | | |
| Example 4 | TSR | 53.6% | 35.0% | Eudragit | 5.0% | Oleyl | 3.0% | 3.4% | 23.28 | 703 | Δ |
| | | | | S100 ⋆ | | alcohol # | | | | | |
| Example 5 | TSR | 53.6% | 35.0% | Eudragit | 5.0% | Isostearyl | 3.0% | 3.4% | 25.25 | 858 | ◯ |
| | | | | S100 ⋆ | | alcohol # | | | | | |
| Example 6 | TSR | 53.6% | 35.0% | Eudragit | 5.0% | Lauryl | 3.0% | 3.4% | 21.15 | 786 | ◯ |
| | | | | S100 ⋆ | | alcohol # | | | | | |
| Comparative | Duro-Tak87- | 61.6% | 35.0% | — | | — | | 3.4% | 9.40 | 1032 | X |
| example 1 | 4098 | | | | | | | | | | |
| Comparative | Duro-Tak87- | 56.6% | 35.0% | Eudragit | 5.0% | — | | 3.4% | 7.91 | 933 | X |
| example 2 | 4098 | | | EPO * | | | | | | | |
| Comparative | Duro-Tak87- | 67.1% | 30.0% | — | | — | | 2.9% | 10.47 | 912 | X |
| example 3 | 2516 # | | | | | | | | | | |
| Comparative | Duro-Tak87- | 61.6% | 35.0% | — | | — | | 3.4% | 13.34 | 913 | X |
| example 4 | 2516 # | | | | | | | | | | |
| Comparative | Duro-Tak87- | 56.6% | 35.0% | Eudragit | 5.0% | — | | 3.4% | 11.41 | 1066 | X |
| example 5 | 2516 # | | | EPO * | | | | | | | |
| Comparative | Duro-Tak87- | 58.6% | 35.0% | — | | Pyrothio- | 3.0% | 3.4% | 8.93 | 610 | X |
| example 6 | 2516 # | | | | | decane | | | | | |
| Comparative | Duro-Tak87- | 58.6% | 35.0% | — | | GML # | 3.0% | 3.4% | 11.46 | 980 | Δ |
| example 7 | 2516 # | | | | | | | | | | |
| Comparative | Duro-Tak87- | 58.6% | 35.0% | — | | PGML # | 3.0% | 3.4% | 8.28 | 791 | X |
| example 8 | 2516 # | | | | | | | | | | |
| Comparative | Duro-Tak87- | 58.6% | 35.0% | — | | Isostearyl | 3.0% | 3.4% | 8.70 | 469 | Δ |
| example 9 | 2516 # | | | | | alcohol # | | | | | |
| Comparative | TSR | 61.6% | 35.0% | — | | — | | 3.4% | 12.38 | 1080 | X |
| example 10 | | | | | | | | | | | |

⋆: Carboxyl-group-containing compound
* Amino-group-containing compound
#: Hydroxyl-group-containing compound
Judgment criteria of crystallization
◯: No crystal
Δ: Slight crystallization
X: Crystallization on the half to entire surface

### Test Example 1

### Hairless Mouse Skin Permeation Test

Hairless mouse dorsal skin was peeled off and mounted on a flow-through cell (5 cm²) with the dermis side positioned on the receptor layer side, through the outer periphery of which flow-through cell warm water at 37° C. was circulated. Each of the preparations obtained in Examples 1-6 and Comparative examples 1-10 (7 days after production) was adhered to the stratum corneum side, and sampling was carried out using PBS of pH7.4 on the receptor layer at 5 ml/h every 2 h up to 24 h. The drug concentration in the receptor solution obtained at each sampling time was measured by high-speed liquid chromatography, its flow rate being precisely measured. Permeation rate per 1 h was calculated from the measured values of the flow rate and drug concentration, and maximum skin permeation rates (Jmax (μg/cm²/hr)) of the preparations obtained in Examples 1-6 and Comparative examples 1-10 were determined. As the maximum skin permeation rate increases, the preparation is regarded to have better transdermal absorption properties. Table 1 shows the results.

From the results shown in Table 1, it is understood that all of the adhesive patches of the invention (Example 1-6), compared to the adhesive patches of Comparative examples 1-10, have the superior maximum skin permeation rate (Jmax) with respect to the basic drug (donepezil) that has the logP value in the free state of 3 or more. In addition, from the comparisons between Example 1 and Comparative example 1, between Example 2 and Comparative example 3, or between Example 3 and Comparative example 10, it was clarified that the addition of the (meth)acrylate copolymer having a carboxyl group (Eudragit S100 or L100) improved skin permeability by as much as approximately 30-60% compared to the cases without the addition. Furthermore, when the (meth)acrylate copolymer without a carboxyl group (aminoalkyl methacrylate copolymer (Eudragit EPO)) was added (Comparative examples 2 and 5), such an improvement of the skin permeability was not observed, in contrast the skin permeability being decreased compared to the cases with no addition thereof (Comparative examples 1 and 4); thus, it is to be understood that the improvement of the skin permeability by (meth)acrylate copolymers is specific to the (meth)acrylate copolymers having a carboxyl group.

The result also indicated that addition of an absorption enhancer such as an aliphatic alcohol (oleyl alcohol (Example 4), isostearyl alcohol (Example 5), or lauryl alcohol (Example 6)) in addition to the essential components of the invention (a basic drug having the logarithm of octanol-water partition coefficient in the free state of 3 or more, an adhesive base, and a (meth)acrylate copolymer having a carboxyl group) improved the skin permeability of the inventive patch. In contrast, blending an absorption enhancer without a (meth)acrylate copolymer (Comparative examples 6-9), the absorption enhancer showed no improvement of transdermal absorption properties: The skin permeability significantly decreased compared to the cases without an absorption enhancer. As such, it was shown that the improvement of transdermal absorption properties due to an absorption enhancer cannot be obtained by the absorption enhancer alone, but the improvement can be realized only when the absorption enhancer is blended into the adhesive layer together with a (meth)acrylate copolymer.

### Test Example 2

### Physical Property (Adhesive Force) Test of Preparations

With respect to the preparations obtained in Examples 1-6 and Comparative examples 1-10, their adhesive forces were measured by a probe tack tester. Table 1 shows the results.

While preferable adhesive force of transdermal absorption patches is 600-1000 as a tack value (gF), the results of Table 1 show that all of the tack values of the adhesive patches of the invention (Examples 1-6) are within this range, indicating that the inventive patches have excellent adhesive force. In addition, in the adhesive patch of Comparative example 9 which contains isostearyl alcohol as an absorption enhancer, the adhesive layer was plasticized by the addition of the isostearyl alcohol, resulting in a significant decrease in the adhesive force. However, the adhesive patch of Example 5 which contains isostearyl alcohol and the (meth)acrylate copolymer having a carboxyl group has a good adhesive force; thus, plasticization of adhesive layers due to absorption enhancers such as an aliphatic alcohol was shown to be prevented by blending a (meth)acrylate copolymer having a carboxyl group.

### Test Example 3

### Preparation Stability Test

With respect to the preparations obtained in Examples 1-6 and Comparative examples 1-10, presence/absence of crystallization was observed up to 30 days after production. Table 1 shows the results.

As clearly understood from the results shown in Table 1, while no crystal deposition or only slight crystallization was observed in the adhesive patches of the invention (Example 1-6), crystal deposition was observed on the almost half to the entire surface of the adhesive patches of Comparative examples 1-10, indicating that the latter patches were unable to keep the drug in a stable dissolved state. Therefore, it was shown that, by blending a (meth)acrylate copolymer having a carboxyl group in the adhesive layer, even when a basic drug (for example, donepezil) hardly soluble in generally-lipophilic adhesive bases is used, the basic drug can be kept in the adhesive layer at a high concentration of 30 wt % or more in a stable dissolved state for a long period after production of preparations.

Thus, as mentioned above, it was shown that due to the (meth)acrylate copolymer having a carboxyl group used in the invention, an adhesive patch wherein a basic drug (for example, donepezil), which is low soluble in oil with a logP value in the free sate of 3 or more and hardly soluble in water, is dissolved in the adhesive layer at a high concentration (such as 30 wt % or more) can be realized; and the transdermal absorption properties of the basic drug (for example, donepezil) having a logP value in the free sate of 3 or more can be markedly improved by the above adhesive patch. In addition, it is clearly understood that the adhesive patch of the invention wherein a (meth)acrylate copolymer is contained in the adhesive layer together with a basic drug (for example, donepezil) having a logP value in the free sate of 3 or more shows superior skin permeability and good stability of the medicinal ingredients in the preparation, and can continually exert its drug efficacy. Moreover, it is clearly understood that in the adhesive patch of the invention containing a (meth)acrylate copolymer by blending an absorption enhancer such as oleyl alcohol, isostearyl alcohol or lauryl alcohol, the adhesive patch having more superior skin permeability without deteriorating the adhesiveness as adhesive patches can be provided. Furthermore, it is clearly understood that in such adhesive patches, by the use of an acrylic adhesive agent as an adhesive, the adhesive patch having remarkably superior transdermal absorption properties and drug-efficacy sustainability can be provided. Moreover, it was shown that, if formulated as an adhesive patch containing donepezil, the inventive preparation has above-mentioned effects with remarkably superior transdermal absorption properties, as well as excellent physical properties and stability.

### INDUSTRIAL APPLICABILITY

As explained above, according to the invention, it becomes possible to provide a transdermal absorption patch, which can dissolve even a basic drug that has a low solubility in adhesive bases into the adhesive layer at a high concentration, and which has remarkably superior transdermal absorption properties of the drug, excellent physical properties, and excellent time course stability of the drug in the preparation; therefore, the adhesive patch of the invention is expected to be applied as a pharmaceutical agent that can simplify the dosing method and improve compliance. Moreover, the invention can provide a donepezil-containing transdermal absorption patch with superior transdermal absorption properties that can be used for the therapy of patients.

1. A transdermal absorption patch containing a basic drug having a logarithm of octanol-water partition coefficient in the free state of 3 or more, an adhesive base, and a (meth)acrylate copolymer having a carboxyl group.

2. The transdermal absorption patch according to claim 1, wherein the adhesive base is an acrylic adhesive.

3. The transdermal absorption patch according to claim 2, wherein the acrylic adhesive is a polymer having a carboxyl group or a hydroxyl group.

4. The transdermal absorption patch according to claim 1, wherein the (meth)acrylate copolymer having a carboxyl group is a copolymer comprising methacrylic acid and methyl methacrylate.

5. The transdermal absorption patch according to claim 1, further containing a fatty acid and/or an aliphatic alcohol having a carbon number of 8 or more.

6. The transdermal absorption patch according to claim 1, wherein the basic drug has a low oil solubility.

7. The transdermal absorption patch according to claim 1, wherein the basic drug has a low water solubility.

8. The transdermal absorption patch according to claim 1, wherein the basic drug is donepezil.

9. The transdermal absorption patch according to claim 1, wherein an amount of the basic drug is 5 wt % or more relative to a total weight of the adhesive layer.

10. A method of treating a patient, comprising: treating the patient with a transdermal absorption patch of claim 1.

11. The method of claim 10 wherein a drug is administered to the patient transdermally via the patch.

12. The method of claim 10 wherein donepezil is administered to the patient via the patch.

13. The method of claim 11 wherein donepezil is administered to the patient via the patch.
